(19) United States
(12) Patent Application Publication
(10) Pub. No.: US 2007/0179803 A1
(43) Pub. Date: Aug. 2, 2007

(54) Method for verifying the feasibility of a medical study using acceptance criteria for patients

(76) Inventors: Klaus Abraham-Fuchs, Erlangen (DE); Rainer Kuth, Herzogenaurach (DE); Eva Rumpel, Erlangen (DE); Markus Schmidt, Nurnberg (DE); Siegfried Schneider, Erlangen (DE); Horst Schreiner, Furth (DE); Gudrun Zahlmann, Neumarkt (DE)

Correspondence Address: HARNESS, DICKEY & PIERCE, P.L.C., P.O.BOX 8910, RESTON, VA, 20195, US

(21) Appl. No.: 10/589,562

(22) PCT Filed: Jan. 31, 2005
(86) PCT No.: PCT/EP05/50401

§ 371 Date: Aug. 16, 2006

(30) Foreign Application Priority Data
Feb. 18, 2004 (DE) 10 2004 008 189.1
Oct. 28, 2004 (DE) 10 2004 052 547.1

(51) Int. Cl. G06Q 10/00; A61B 5/00

(52) U.S. Cl. 705/2; 600/300

(57) ABSTRACT

A method is for verifying the feasibility of a medical study using acceptance criteria for patients. The method includes defining target criteria for the study. Based on the acceptance criteria, a patient collective including potential patients is selected from a database containing patient data of patients. The patient data of the patient collective are evaluated based on the target criteria. Finally, a measure for the feasibility of the study is established.

### PRIORITY STATEMENT

This application is the national phase under 35 U.S.C. § 371 of PCT International Application No. PCT/EP2005/050401 which has an International filing date of Jan. 31, 2005, which designated the United States of America and which claims priority on German Patent Application numbers 10 2004 008 189.1 filed Feb. 18, 2004 and 10 2004 052 547.1 filed Oct. 28, 2004, the entire contents of which are hereby incorporated herein by reference.

### FIELD

The invention generally relates to a method for verifying the feasibility of a medical project with acceptance criteria for patients.

### BACKGROUND

Medical projects which have acceptance criteria for patients participating in then are, for example, in-house outcome analyses of a pharmaceutical company or technology assessments for evaluating medical techniques, but primarily clinical studies. Such projects are commissioned by various institutions such as pharmaceutical companies, clinics or state bodies, in order to test new medicaments, therapy methods, treatment methods etc. on patients. The aim is evaluation, assessment or approval of the tested product before an official institution.

Testing is carried out on a patient group, including a few to several thousand patients. In order to obtain valid information from such a project, the patients must satisfy very particular properties so as to form a comparable basis for the project. The properties are set down in the form of acceptance criteria in a so-called patient profile. The patient profile is part of a protocol, i.e. a description of the project, which is set before it starts to be conducted and describes the entire project. All properties which a patient may have could be envisaged as acceptance criteria for patients. These are, for example, age, sex, disease/diagnosis or other diseases of the patient, but also the geographical region or social class from which the patient comes.

Such a protocol can no longer be modified after starting to conduct the project, or can be modified only insubstantially. Changes are not possible for various reasons. For example, the protocol must be filed with an authority since the approval of a new medicament depends on this. Furthermore, data obtained in the scope of the project before and after changing the protocol are not necessary comparable. Once the project is underway, the protocol must therefore remain unchanged in respect of the acceptance criteria and binding for all those involved, i.e. those conducting it and the patients. The patient profile must therefore also be set before the start of the project.

In the course of such a project, it is then often problematic to find a sufficient number of patients who fulfill the patient profile or the acceptance criteria in a sufficiently short time after its start.

If enough patients cannot be found for the patient type specified in the patient profile, the project generally can only be stopped. This usually means significant financial and time losses for the sponsor. Such termination is particularly annoying when just minor changes in the patient profile, which are insignificant for the validity or meaningfulness of the project but for example are inconceivable after its start, would be sufficient in order to achieve an increased number of patients more rapidly and thus still be able to continue the project.

[PHARSIGHT, “Trial-Simulator product brochure, Mountain-view, USA, 2002] discloses a simulation program for clinical studies, which simulates the running of a study based on biochemical and medical model calculations with the aid of a theoretical patient group. In this way, for example, it is possible to discover such implausibilities in the study protocol which conflict with known medical discoveries. The quality of the verification of a study protocol by such a simulation, however, depends strongly on the model quality of the patient model in the simulation program.

Although such modeling provides information about an imaginary patient group, it does not provide any information about genuinely existing patients actually present for example in a particular region or at a particular time, or actually available for the study. Verification of the feasibility of the project can therefore be carried out only to a limited extent with such an aid.

### SUMMARY

At least one embodiment of the present invention improves the verification of the feasibility of a medical project with acceptance criteria for patients.

A method, in at least one embodiment, is for verifying the feasibility of a medical project with acceptance criteria for patients. The method includes: target criteria are set for the project. A patient group comprising potential patients is selected with the aid of the acceptance criteria from a database containing patient data of patients. The patient data of the patient group are evaluated with the aid of the target criteria and a measure of the feasibility of the project is determined.

The acceptance criteria allocated to the project filter out the set of patients fulfilling the acceptance criteria, who can be envisaged as potential patients for the project, from the set of all patients. The actual participation of a potential patient in the project then naturally depends on their personal consent.

Target criteria for the project are the criteria which describe the question of feasibility of the project. This is usually the number of patients who actually participate in the full term of the project. Further criteria may nevertheless also be envisaged, for example how high the dropout rate of the study participants in the course of the project may be, from which region the patients should come, whether the patients have health insurance etc. Such target criteria must necessarily be satisfied during or after the end of the project since, for example, the approval of a new medication depends on this or only in this way is it possible to obtain reliable conclusions about the subject-matter of the project owing to statistical considerations.

The database contains patient data of genuinely existing patients; for example, electronic patient files, databases of clinics, medical practices or non-medical databases to which patients are assigned, for example a local government office for the registration of residents, a health insurance company or a data warehouse may be envisaged.

Since the data stored in the databases are assigned to genuinely existing patients, i.e. they are not just theoretical data as used in models, this represents an accurate picture of reality. The information obtained with the aid of these data always corresponds to reality.

The assignment of patients to data can be carried out directly if the data are stored as clear data, and by authorized individuals for pseudonymized data, but it is not possible for anonymized data. Depending on the database, data are medical data, socio-economic data or generally person-related data.

Via database querying of said database, information which is realistic and up to date at the time of the query is thus determined about a genuinely existing patient population.

The databases are situated at one location or are distributed over several locations. A database query may be performed via a central database query, a special browser which accesses distributed databases, or other techniques such as software agents. In the case of distributed databases, for example at different clinics potentially suitable for a clinical study, such a software agent could be installed at each clinic in the database there and, when interrogated or if a particular pattern occurs in the database, could send patient data to a remotely located central computer which is set up for checking the feasibility of the project, for example at a pharmaceutical company as the sponsor.

From all patients available through the database, those who fulfill the acceptance criteria are determined by the database query. They then form the patient group, i.e. the set of all real patients who are potentially suitable for the medical project.

With the patient data of the potentially suitable patients, the database query delivers miscellaneous additional information about this patient group which is subsequently evaluated with the aid of the target criteria. If the target criterion is the participant number, for example, it is possible to establish how many patients are available as potential participants for the project. It is now possible to determine virtually any extra information from the additional data in the patients data records, for example what percentage of the patient group may be expected as actual participants in the project. It is also possible, for example, to determine the local distribution of the patients, their social structure, work habits, whether they are invalided or have health insurance, occurrence of other diseases, likelihood of moving away from where the project is conducted, financial structure or expected mortality rate.

Since the verification is based on data of genuinely existing patients, it is directly applicable to reality and is therefore more accurate than a verification based on model calculations. The verification is practical. The feasibility of the clinical study can be verified with any desired accuracy by using diversified databases and precisely specified target criteria.

As a measure of the feasibility of the project, for example, it is thus possible to calculate a simple Yes/No appraisal or a likelihood that it will be carried out successfully.

Owing to the wealth of information which can be determined from the patient data, risks and contraindications which endanger the conduct of the project can be determined already before carrying it out, for example frequently occurring other diseases or above-averagely high mortality of the patient group specified by the acceptance criteria.

Supplementary information for the project may also be determined by evaluating the patient data. For instance, the local distribution of a patient group suitable for a project may lead to a favorable choice of where the project is conducted, which for example ensures that the patients can more rapidly reach the place where it is conducted so that the fewer patients will be expected to stop their participation in the project because of long journeys.

The acceptance criteria may be modified with the aid of the patient data of the patient group. To this end, it is possible to use the information determined from the patient data of the patient group which, for example, already provides indications of a more favorable modification of the acceptance criteria in respect of the target criteria. If the analysis of the patient data shows that most patients with a disease to be studied work in a particular factory, for example, the acceptance criterion “entry age” could be adapted to the staffing structure of the factory so as to obtain more patients as potential participants, since it is to be expected that many of the factory workers are sick.

Potential study participants are again selected by a new query from the database with the modified acceptance criteria, which generally leads to a modified patient group because of the different acceptance criteria. The patient data of the patient group now newly determined are again evaluated with the aid of the target criteria, and a new measure of the feasibility of the project is determined. It is possible to optimize the acceptance criteria by iteratively carrying out database queries with varying acceptance criteria, the feasibility being verified with the aid of genuinely existing patients each time. The concept of the project, for example in the form of acceptance criteria, can thus be adapted optimally to the genuinely existing patient structure, which virtually ensures the feasibility.

By the iterative method, it is possible to identify which changes in the design cause which modifications in the patient group, so that a suitable or even optimal patient group can be found for the project in a rapid, simple and cost-effective way.

A patient model may be compiled with the aid of the patient data of the patient group, and modified acceptance criteria may be created with the aid of the patient model. If particular properties of the patients reflected by the database can be modeled, for example the age structure of the patients recorded in the database, then these influences can be used in the form of a model for verifying the feasibility of the project and need not in respect of this aspect be determined by frequent iterative database queries. The database queries can thus be restricted to non-modelable aspects of the patient data, so that the database queries are accelerated and the number of iteration steps is reduced. Since the modeling is based on the patient data of genuinely existing patients, it is more accurate than purely theoretical modeling based on biological or medical theories.

The patient data of the patient group may be used for actually carrying out the project. The data determined during verification of the feasibility thus fulfill a twofold purpose and are not only used for the verification, but are also used for or in the conduct of the project. In the case of clear data or pseudonymized data, for example, the identities of potential participants may be determined from the database and they may be personally asked directly, for example written to, whether they wish to participate in the study. In the case of anonymized data, for example, advertising campaigns for a project may be geographically limited on the basis of socio-economic data and carried out in a targeted way through suitable media, which saves time, work and costs for the sponsor of the study. The planning and conduct of the next working steps, such as publicity work, patient interviews, creation of training material etc. is accelerated and simplified in the scope of the clinical study.

### BRIEF DESCRIPTION OF THE DRAWINGS

For a further description of the invention, reference will be made to the example embodiments of the drawing in which, in a schematic representation:

FIG. 1 shows a flow chart of a method for verifying the feasibility of a clinical study.

### DETAILED DESCRIPTION OF THE EXAMPLE EMBODIMENTS

Since there is a consistent terminology with respect to clinical studies and the aspects of said medical projects can be translated into it, embodiments of the invention are explained with the aid of a clinical study even though this is also intended to include other equivalent medical projects. If the project is a clinical study, the acceptance criteria are the inclusion/exclusion criteria set in the study protocol, which characterize a patient as a suitable participant for the study.

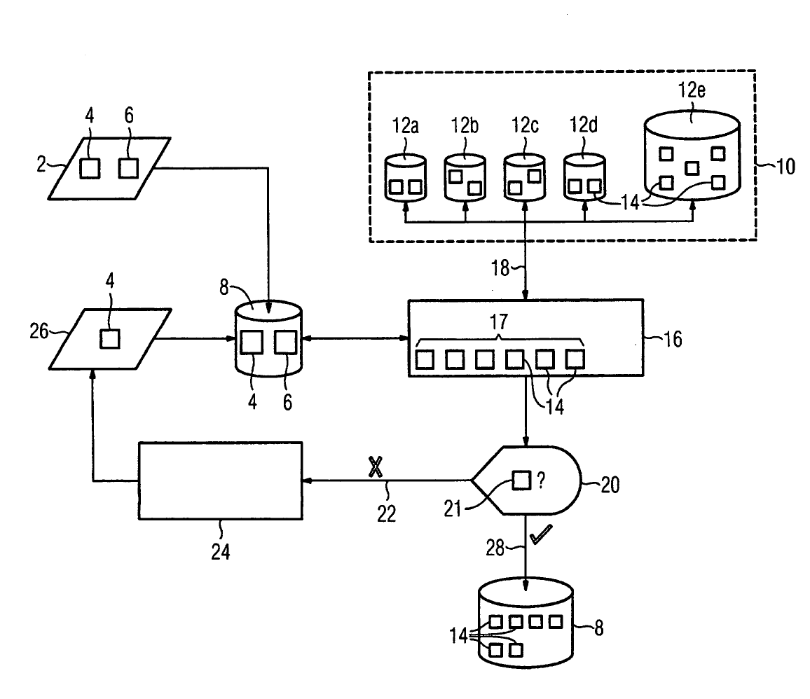

FIG. 1 describes the procedure of verifying the feasibility of a clinical study. The verification is carried out by a pharmaceutical company as the backer or sponsor of the study. The purpose of the study is to test a new medicament in a state approval process. The official approval requirements are that the data should be evaluated by at least 50 patients who participate in the study throughout the course of the study.

The pharmaceutical company additionally decides to limit the study to two conduct sites/centers for cost reasons. The target criteria of the study are thus set.

During the development of the study, the study design, a patient profile was developed for participating patients. The verification described below is now intended to establish whether at least 50 study participants are available for the given patient profile. It is therefore possible to decide whether the study can be carried out or whether the patient profile must be modified, and finally leads to a sufficient patient number by modification.

In a start step 2, the acceptance criteria 4 developed in the study design are put into an electronically processable form. The acceptance criteria 4 for patients in this case read: “aged between 40 and 75 years, suffer from diabetes and high blood pressure, blood group O”.

The target criteria 6 for the clinical study to be carried out are likewise put into an electronic form in the start step 2. The target criteria 6 of the study are: “the minimum number of study participants is 50, the study is carried out at most at 2 different institutions”.

The acceptance criteria 4 and target criteria 6 are stored in a database 8. The database 8 is part of a data-processing system (not shown) of the pharmaceutical company, which carries out the verification electronically.

FIG. 1 represents the database pool 10 of a data warehouse in which database queries can be carried out for payment. The database pool 10 contains the patient databases 12a-d of four smaller hospitals and the patient database 12e of a large clinic. The patient databases 12a-e contain patient data 14 of patients currently treated in the relevant clinics.

In an evaluation step 16, the acceptance criteria 4 are formulated in the form of a database query and, as symbolized by the arrow 18, sent to the databases 12a-e where the patient data 14 are searched through with the aid of the acceptance criteria 4. As a result of the database query, the database pool 10 returns patient data 14 which are assigned to patients who fulfill the acceptance criteria 4 for the clinical study.

The patients assigned to the patient data 14 thus form the patient group 17 of all patients potentially suitable for the study. In this context, potentially refers to the fact that the patients must naturally grant their consent before in fact finally participating in the study. The patient data 14 contain all information available in the data warehouse about the patients, inter alia including data of local government offices for the registration of residents.

In the test step 20, the patient data 14 determined in the evaluation step 16 are now evaluated in respect of the target criteria 6. The evaluation reveals the following: 30 patients are available at the clinic assigned to the database 12a, 14 at the clinic corresponding to the database 12c and 72 patients in the large clinic, all of whom fulfill the acceptance criteria 4. According to experience, 50% of suitable patients actually participate in a study. This information is entered by the sponsor into an evaluation system (not shown). A further evaluation of the patient data 14 reveals: the mortality rate to be expected in the patients is 5% during the conduct phase of the clinical study.

If the clinic of the database 12a and the large clinic (12e) are selected as study sites, then a patient count of approximately 48.5 is obtained for patients who participate in the entire study. The percentage expected participant number is now calculated against the aimed participant number of 50 as a measure 21 of the feasibility of the study. The value 97% is thus obtained for the measure 21. The target criteria 8 are therefore not fulfilled with respect to the minimum number of patients, which corresponds to a participant number of at least 100%. At least one of the target criteria 8 for the clinical study is thus not fulfilled, so that the NO decision 22 is made in the flow chart according to FIG. 1, which leads to a modification step 24.

In the modification step 24, the results of the test step 22 are analyzed by the persons in charge of the study design, and the acceptance criteria 4 are reconsidered. From the results determined so far, it is clear that widening the age range for study participants could lead to a sufficient patient number. In a restart step 26, modified acceptance criteria 4 are therefore created. Merely a minor change is carried out since the target criterion 6 relating to 50 patients was only just missed, i.e. the maximum age is increased from 75 to 76 years in the “age” acceptance criterion 4.

The modified acceptance criteria 4 are in turn stored in the database 8, and the method procedure described above is carried out again. Owing to the changed acceptance criteria 4, the database query 18 delivers modified patient data 14 with a modified patient group 17 in the evaluation step 16. The test step 20 reveals that only one more patient is available in the end, so that the target criteria 6 are still not yet fulfilled with a measure 17 of 99%.

For a third run of the method, the age range of the patients is therefore changed to 39-75 years in the modification step 24, and the entire method is carried out again as described above.

This time, the test step 20 reveals that 54 suitable patients remain so that the YES decision 28 is made in the test step 20 owing to the measure 17 of 108%, and the patient data 14 of all patients now suitable in the patient group 17 are transferred into the study database 8. The decision is made to carry out the study, and the study is started.

The patient data 14 are now available for further processing in the study database. An analysis of the patient data 14 reveals: the database 12a of the hospital is stored in pseudonymized form. By sending the pseudonymous patient identifications to the clinic, the latter is capable of writing directly to the relevant patients and inviting them to participate in the clinical study.

The database 12e of the large clinic is stored in anonymized form. Direct access to the selected potential patients is thus not possible. An analysis of the patient data 14 reveals that a large proportion of the patients are employed in a plant located near the large clinic, whereupon an advertising campaign for the clinical study is started in this plant. Many relevant patients furthermore live in a residential area of very small size, so that a target-oriented mailshot which is inexpensive for the sponsor is started there.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for calculating a measure of the feasibility of a medical project with acceptance criteria for patients, comprising:
setting target criteria for the project;
selecting a patient group including potential patients, with the aid of the acceptance criteria, from a database containing patient data of the patients; and
evaluating the patient data of the patient group, with the aid of the selected target criteria, and calculating the measure of the feasibility of the medical project.

2. The method as claimed in claim 1, wherein:
the acceptance criteria are modified with the aid of the patient data of the patient group.

3. The method as claimed in claim 1, wherein:
a patient model is compiled with the aid of the patient data of the patient group; and
the acceptance criteria are modified with the aid of the patient model.

4. The method as claimed in claim 1, wherein:
the patient data of the patient group are used for carrying out the medical project.

5. The method as claimed in claim 1, wherein:
a clinical study is verified as the medical project.

6. The method as claimed in claim 2, wherein:
a patient model is compiled with the aid of the patient data of the patient group; and
the acceptance criteria are modified with the aid of the patient model.

7. The method as claimed in claim 2, wherein:
the patient data of the patient group are used for carrying out the medical project.

8. The method as claimed in claim 3, wherein:
the patient data of the patient group are used for carrying out the medical project.

9. The method as claimed in claim 6, wherein:
the patient data of the patient group are used for carrying out the medical project.

10. The method as claimed in claim 2, wherein:
a clinical study is verified as the medical project.

11. The method as claimed in claim 3, wherein:
a clinical study is verified as the medical project.

12. The method as claimed in claim 4, wherein:
a clinical study is verified as the medical project.
